(19) United States
(12) Patent Application Publication
(10) Pub. No.: US 2008/0200928 A1
(43) Pub. Date: Aug. 21, 2008

(54) Support for Steadying a Surgical Tool

(76) Inventors: Joan Savall Calvo, San Sebastian (Guipuzcoa) (ES); Manuel Manrique Rodriguez, Pamplona ( Navarra) (ES)

Correspondence Address: MOORE & VAN ALLEN PLLC, P.O. BOX 13706, Research Triangle Park, NC, 27709, US

(21) Appl. No.: 11/718,440

(22) PCT Filed: Oct. 7, 2005
(86) PCT No.: PCT/EP2005/010902

§ 371 Date: Jun. 26, 2007

(30) Foreign Application Priority Data
Nov. 4, 2004 (ES) 200402721

(51) Int. Cl. A61B 19/00

(52) U.S. Cl. 606/130

(57) ABSTRACT

Support or micromanipulator (100) for a surgical tool (20) comprising a complaint mechanism (1) providing direct support to the surgical tool (200), a positioning mechanism giving support to said complaint mechanism, and an attachment mechanism giving support to said positioning mechanism. The complaint mechanism (1) is made of a complaint material and allows three or four degrees of freedom to the movement of the tool (200). The positioning mechanism is located between the attachment mechanism and the complaint mechanism (1) and allows positioning of the same with six degrees of freedom. The attachment mechanism fastens the assembly to a surface of the surgical intervention region. The micromanipulator (100) is especially useful for practicing surgical interventions in the cochlea (performing cochlear implants, obtaining samples, etc), or other interventions for which high precision with the tool (200) is required.

The present invention relates to a support for steadying a surgical tool handled by a surgeon which comprises a limiting means for limiting the movement of said tool.

### BACKGROUND OF THE INVENTION

The inner ear is located inside the temporal bone, the latter also containing the hearing and equilibrium organs, which in turn contain sensitive hail cells connected to the auditory nerve. The inner ear is separated from the middle ear by the oval window and is constituted by a number or membrane channels housed in the a dense portion of the temporal bone. More precisely the inner ear is composed of the cochlea, the vestibuli and three semicircular channels. These structures communicate and contain two types of fluid, the endolymph and the perilymph.

The sound waves are transmitted through the ear canal and then impinge the ear drum causing it to vibrate. These vibrations are then transmitted to the three small bones (malleus, incus and stapes) and through the oval window reach the fluids in the inner ear. The movements of the endolymph and the perilymph excite the hair cells, constituting the Organ of Corti, said cells transmitting neural signals directly to the auditory nerve, along which said signals reach the cortex where they are processed in specialized areas of the brain. The response pattern of the hair cells codifies the acoustic information so that it can be interpreted by the auditory nuclei of the brain.

The diseases which affect the Organ of Corti are responsible for most cases of audition loss. When these losses are severe they can be treated with an electronic device called cochlear implant. This device converts acoustic waves in electric signals released through a number of electrodes implanted in the cochlea, so that the auditory nerve can be directly excited.

In the surgical intervention needed to place a cochlear implant a small aperture is drilled through the bone behind the ear and a groove is milled in the mastoid region of the temporal bone. This permits placing the internal part of the implant and securing the electrodes in the cochlea.

A new type of flat electrode carrier have been developed which is inserted between the bone and the spiral ligament, located in the side wall of the cochlea. In order to place this new type of implant it is necessary to mill a groove on a protrusion of the temporal bone, just to uncover the cochlea spiral ligament so that the flat electrode carrier can be inserted between the ligament and the bone. This operation is very arduous and, hence, it is difficult for a surgeon to successfully complete it without damaging the cochlea, relying just upon his/her skill.

For the sake of steadying and stabilizing the surgical tools employed manually by a surgeon in surgical interventions of high accuracy different kinds of supports are known.

Patent U.S. Pat. No. 4,993,406 describes a stabilizing support for surgical instruments configured for use with a speculum, said support including a spring-mounted bridge having a plurality of grooves in which a surgical instrument rests during microsurgery.

Patent U.S. Pat. No. 5,201,742 discloses a jig for orientating surgical instruments which has a base for placement against a tissue in the region of surgery, a yoke, an instrument support sleeve on the yoke, a structure for supporting the yoke on the base and a structure for maintaining the yoke in a plurality of selected positions relative to the base. This support jig is mainly applied to laparoscopy.

Supports of the kind described in these patents cannot be applied to the high accuracy microsurgery required for interventions in the inner ear, like that performed for placing cochlear implants, taking of samples, local administering of drugs or implanting cells or live tissue.

Therefore the known supports don't solve the problem that any surgeon, irrespective of his/her skill, could access the cochlea accurately without causing harm nor, in general, that of reducing suitably the degrees of freedom of the movement of the tool as driven by the surgeon's hand in places where a highly accurate handling of a surgical tool is required.

### SUMMARY OF THE INVENTION

It is an object of the present invention to provide a support or micromanipulator for manually handled surgical tools, such a support effectively limiting the movement of said tools. Another object of the invention is to achieve a scaling down of the movement of the tool tip.

Said support comprises a means limiting the movement of the surgical tool which reduces in at least two the degrees of freedom of the movement of said tool as driven by the surgeon's hand. Since the degrees of freedom of the free rigid solid in the tridimensional space are six, according to this aspect of the invention the degrees of freedom of the support are limited to one, two, three or four. The permitted degrees of freedom are selected with the condition that the surgical intervention may be performed without causing harm.

In an embodiment of the invention, the degrees of freedom allowed by said limiting means are three translations and one rotation.

Preferably, the degrees of freedom allowed by said limiting means are two translations and one rotation, which are the degrees of freedom of the free rigid solid in the bidimensional plane.

Advantageously, said limiting means comprises a compliant mechanism for supporting said tool. This type of mechanism increases the skill of the surgeon, providing him a control over the movement of the tool by reducing its play, which is fundamental for achieving the required accuracy, while keeping the touch for manually handling the tool. By using said compliant mechanism the micromanipulator can be kept simple and inexpensive.

In case of allowing two translations and one rotation, said compliant mechanism can be topologically equivalent to a rigid symmetrical mechanism made of five bars and six hinged joints, which precisely has the above mentioned three degrees of freedom of the rigid solid in the plane.

In an embodiment, said compliant mechanism comprises a central segment having a hole for inserting the tool, and a blocking means for blocking said tool with respect to the compliant mechanism. In this way, for engaging the tool with the compliant mechanism all what is needed is to insert the latter in said hole, regulate its height and block it with said blocking means.

In addition to the compliant mechanism, the support of the invention may comprise a positioning mechanism for positioning said compliant mechanism. Said positioning mechanism is advantageous because for performing the surgical intervention the compliant mechanism ought to be positioned with the whole six degrees of freedom.

In an embodiment, said positioning mechanism comprises a compliant coupling formed as a tubular segment having two sets of slits positioned at 90° firm each other, said compliant coupling allowing two rotations and one translation, a collar which receives and secures said compliant coupling, and a clamp guide which holds said compliant coupling and is fastened to said collar, said clamp guide being provided with six recesses equally spaced at 60°. For mounting this set, said compliant coupling is fastened to said collar, said clamp guide is fastened to said compliant coupling and its orientation is regulated through a number of pins.

In an embodiment, said positioning mechanism comprises a screw-cylinder having three screwed protrusions, equally spaced at 120°, and a nut-knob, so that said screw-cylinder can be inserted into said clamp guide in six different positions equally spaced at 60°, and the axial position of said screw-cylinder is fine-tuned by means of said nut-knob.

Advantageously said positioning mechanism comprises a holder for said compliant mechanism, the latter having two lugs which are inserted in two corresponding slots formed in said holder. This kind of fastening system for the compliant joint mechanism permits to readily change different types of joint mechanisms.

In an embodiment, the support comprises too an attachment mechanism for attaching said positioning mechanism to a surface near the region to perform surgery on, being of great importance for the correct operation of said support that it may keep unaltered its position relative to the small region of the intervention.

In an embodiment, said attachment mechanism comprises a finger which is attached to said surface and to a connector, said collar being also attached to said connector, said attachment mechanism further comprising two short pins, which can be ascended or descended in order to regulate the height of said collar over said connector, and two long pins, which can also be ascended or descended in order to, provide a good seat for said collar on said surface. Thus said attachment mechanism is secured at one point and rests on other two points.

Advantageously, when performing a cochlear implant said surface is a section of the temporal bone.

In general, the surgical tool can be any suitable microsurgical instrument, applied for instance to taking samples, local administering of drugs at selective points, electric stimulation, etc.

Particularly, said surgical tool can be a milling cutter for milling a groove in the temporal bone, or a micropalette or a microneedle engaged to a suction or injection system

### BRIEF DESCRIPTION OF DRAWINGS

For a better understanding of the present invention, embodiments thereof will be described by making reference to the accompanying drawings, in which:

FIG. 1A is a schematic view of the arrangement of the support or micromanipulator of the preferred embodiment, and FIG. 1B is a schematic view of the groove to be performed;

FIG. 2 is a perspective view of different elements of the micromanipulator;

FIG. 3A is a schematic view of the compliant mechanism, FIG. 3B is a schematic view of an equivalent rigid mechanism and FIG. 3C shows the mounting of the tool in the compliant mechanism;

FIG. 4 is an exploded view of a detail of the positioning mechanism;

FIG. 5 shows the possibility of rotation of the positioning mechanism;

FIG. 6 is an exploded view of another detail of the positioning mechanism;

FIG. 6 is an exploded view of another detail of the positioning mechanism;

FIG. 7 is an exploded view of yet another detail of the positioning mechanism;

FIGS. 8A and 8B show the mounting of the compliant mechanism in the positioning mechanism;

FIG. 9 show the attachment mechanism for attaching the micromanipulator to the bone;

FIG. 10 is an exploded view of the complete micromanipulator;

FIG. 11A is a perspective view of another embodiment of the compliant mechanism, and FIG. 11B is a side view thereof.

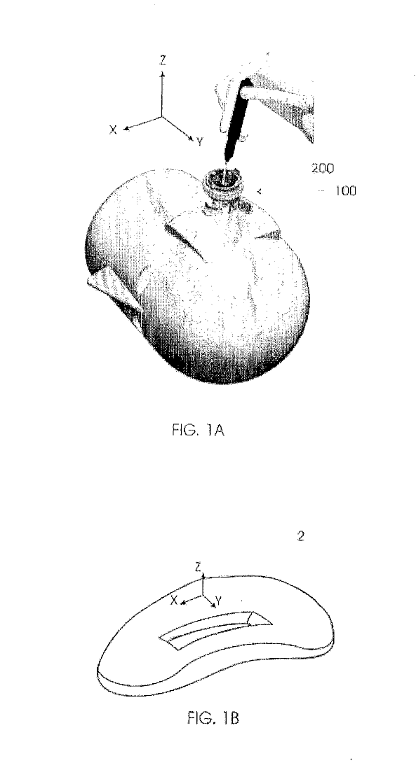

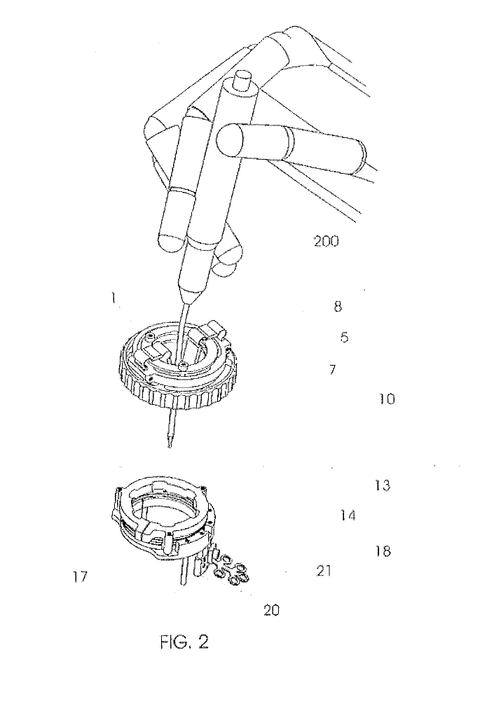

### DESCRIPTION OF PREFERRED EMBODIMENTS

In the following a preferred embodiment of a support or micromanipulator 100 of the invention will be described, which micromanipulator is applied to performing a cochlear implant of a flat electrode carrier. The operation requires milling in the temporal bone a groove 2 approximately 5 mm long, 2 mm wide and 2 mm deep.

Once the groove has been milled and the cochlea spiral ligament has been exposed, the flat electrode carrier has to be inserted between the cochlea and de bone covering it. The implant clamps the cochlea, adjusting to its geometry, along a spiral arc. To insert such a thin flat delicate electrode carrier is a matter of much skill, made still more difficult because of the reduced visibility available to the surgeon once the inserting operation have been started.

For the sake of clarity an orthogonal system of reference XYZ is established, in which axis Z is the ear canal direction, X is the groove main direction and Y is the direction perpendicular to X and Z, i.e., in this system of reference the groove dimensions are x=5 mm, y=2 mm and z=2 mm.

The complete micromanipulator is composed of three components:
Compliant mechanism 1: it provides the direct support to a surgical tool 200.
Positioning mechanism: supports the compliant mechanism.
Attachment mechanism: supports the positioning mechanism.

The compliant mechanism 1 controls and limits passively the tool movement direction and range. It is made of a compliant material and is a mechanism in-which force and movement are transmitted between different elements according to their relative flexibility. In this embodiment the compliant material selected is Aluminium 7075, chosen because of its high elastic limit—elastic modulus ratio, and the compliant mechanism have been made by electroerosion, which is a manufacturing technology of high accuracy which can produce very thin pieces, which is the kind of pieces required if the material itself is to transmit movement.

The compliant mechanism 1 allows three degrees of freedom to the movement of the tool 200, i.e., translation on X an Z and rotation on Y; translation on Z is necessary for milling the groove in depth, translation oil X is necessary for milling the groove longitudinally and rotation on Y is necessary for controlling the incidence angle of the tool and for adapting to the narrow ear canal at any position.

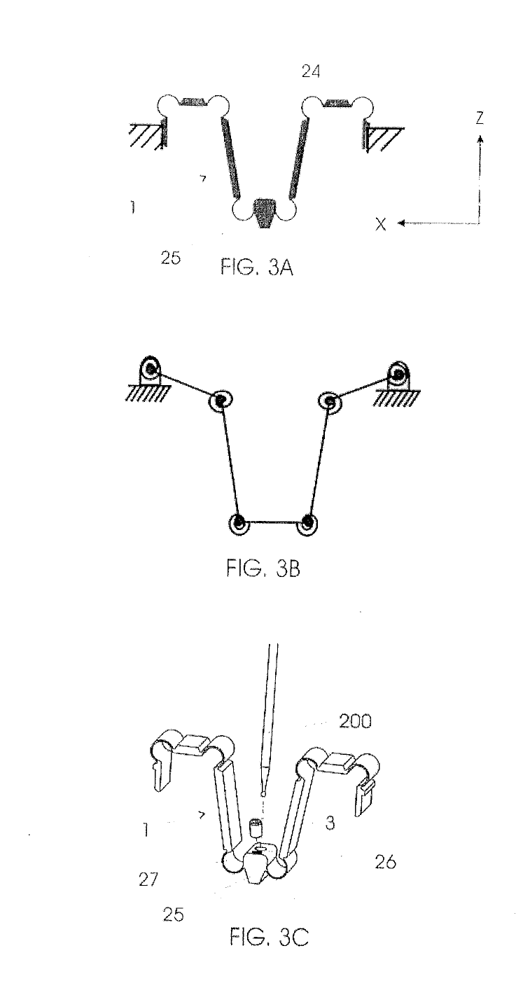

In FIG. 3A can be schematically seen the form of the compliant mechanism 1, which has been designed with the aim that small translations on X and Z and slight rotations on Y shall be relatively easy but that any other movements shall be very difficult.

The mechanism in FIG. 3A is topologically equivalent to a rigid bar mechanism in which the arcs, or knuckles 24, are hinged joints, said rigid mechanism being shown in FIG. 3B. Said symmetric rigid mechanism of five bars and six hinged joints is a known mechanism which provides the three degrees of freedom of a rigid solid in the plane: two translations and one rotation.

For inserting the tool 200 and placing it at a suitable height, the compliant mechanism 1 is provided with a central thicker segment 25 having a hole 26 for inserting the tool. Besides said hole 26 there is a threaded hole 27 which houses a setscrew 3 capable to block the axial movement of the tool 200 relative to the compliant mechanism 1 (FIG. 3C).

In order to have the groove correctly placed it is required to position the compliant mechanism 1 with six degrees of freedom, as it is necessary for the tool 200 to reach the starting position of the groove in a handy and versatile fashion. The positioning mechanism is charged with this task; it is located between the attachment mechanism and the compliant mechanism, and the six degrees of freedom, the six movements thereof being successively performed from the attachment mechanism, are the following: rotation on X and rotation on Y, translation on Z which allows the surgeon to approach the tip of the tool 200 into the groove should the former reach the end of the permitted movement of the compliant mechanism 1 (it is important that this regulation on Z is carried out after the two orientations on X and Y, as this ensures that the translation on Z is effected on an axis Vehicle is also the axis of the tool 200). Then one rotation on Z and two translations on X and Y, respectively, complete the regulation.

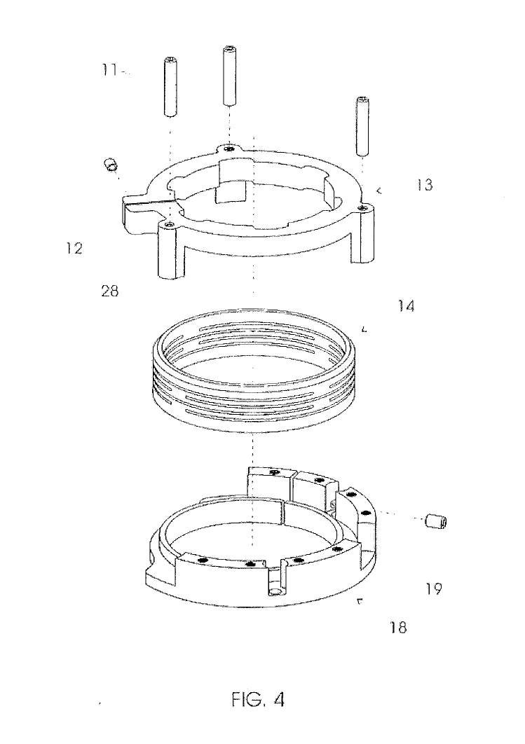

FIG. 4 shows a first component of the positioning mechanism, the function of which is to regulate the orientations on X and Y. To do this said first component is also a compliant device, designed for exhibiting a flexible behavior with respect to the rotations on X and Y and the translation on Z, and a very rigid behavior with respect to the translations on X an Y and the rotation on Z.

Said first component comprises a compliant coupling 14 formed as a tubular segment having two sets of slits, located at 90° from each other on the Z axis, said compliant coupling 14 being made of Aluminium 7075. It also comprises a collar 18 which receives said compliant coupling 14, a setscrew 19 for securing said compliant coupling 14 to aid collar 18, a clamp guide 13 which holds said compliant coupling 14 through a setscrew 12 and is provided with three holes 28, equally spaced at 120°, in which are inserted three setscrews 11 that fasten said clamp guide 13 to said collar 18.

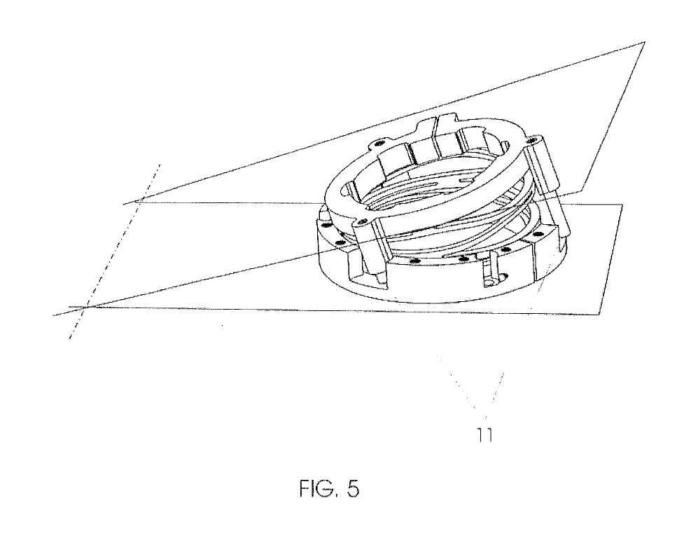

As can be seen in FIG. 5, with the three setscrews 11 both the rotations on X and Y and the translation on Z can be controlled by screwing or unscrewing each of the setscrews 11, and by virtue of the compliant coupling flexibility the clamp guide 13 can be correctly oriented, i.e., its axis can be aligned in the direction of the ear canal. In this embodiment the clamp guide 13 can be tilted up to 15° from the plane XY.

Although with this system for regulating the orientation the depth in Z can also be varied, due to the importance of this movement an additional system has been included which is much more accurate and handy. It must be kept in mind that an error in the depth of the groove 2 could inadmissibly damage the cochlea.

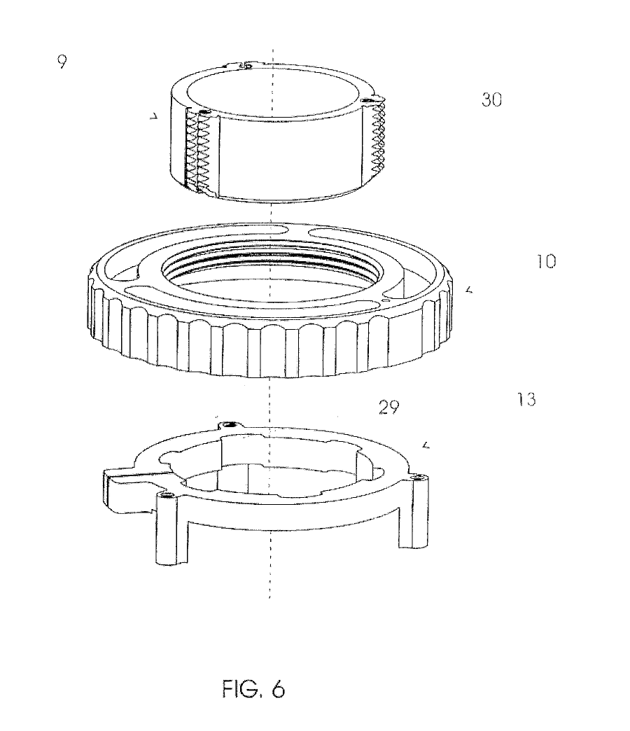

With the aim of fine-tuning the approaching movement of the tool 200 in the Z direction, a further regulation by means of a closely pitched thread have been arranged (FIG. 6). This second component of the positioning mechanism shares with the first component the clamp guide 13 and further comprises a screw-cylinder 9 and a nut-knob 10. The clamp guide 13 is provided with six recesses 29 equally spaced at 60°, and the screw-cylinder 9 is provided with three screwed protrusions 30, equally spaced at 120° which can be loosely inserted in three of said recesses 29, so that said screw-cylinder 9 can be inserted into said clamp guide 13 in six different positions equally spaced at 60°.

The advance on Z of the screw-cylinder 9 is controlled by turning the nut-knob 10, said nut-knob 10 having some reference marks by watching which the advance distance can be read. In this embodiment a closely pitched left thread controls accurately said advance. The movement range of the screw-cylinder 9 on Z is 5 mm.

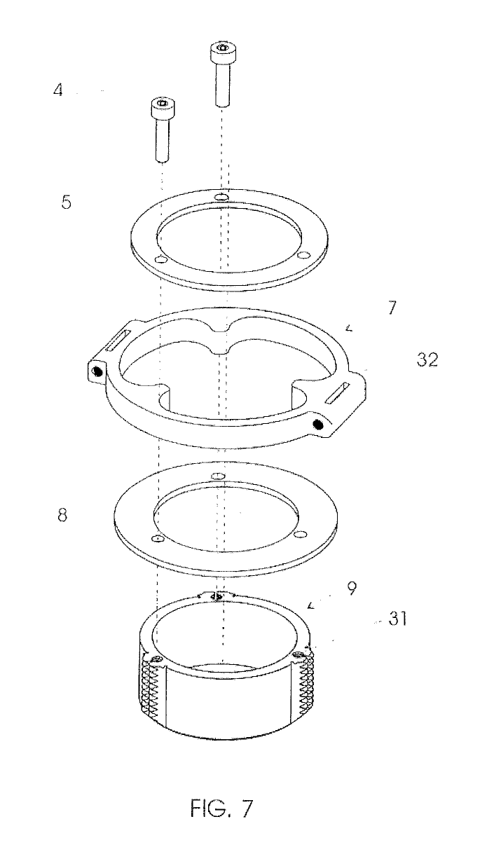

A third component of the positioning mechanism is devoted to setting the linear position with respect to axis X and Y and the angular position with respect to axis Z. Said third component comprises (FIG. 7) a holder 7 for the compliant mechanism 1, an upper washer 5, a lower washer 8 and two bolts 4 which, with the holder 7 laying between the washers 5 and 8, secure these three sandwiched elements to the screw-cylinder 9, by threading in two of a number (three in FIG. 7) of threaded holes 31 made in said screw-cylinder 9.

For setting the position in plane XY, as well as the rotation on Z, all what is needed is to slide the holder 7 between the two washers 5 and 8. In order to do so the bolts 4 must be loosened, the holder 7 must be suitably placed and said bolts 4 must be gently tightened again. The holder 7 is provided, on its inner wall, with three protrusions 32 equally spaced at 120° which act as butts for the bolts 4, in such a way that the rotation of said holder 7 relative to the screw-cylinder 9 can reach 60°. Since said screw-cylinder 9 can be placed in six positions equally spaced at 60° in said clamp guide 13, it turns out that the rotation of holder 7 on Z can go over the 360° of the circumference.

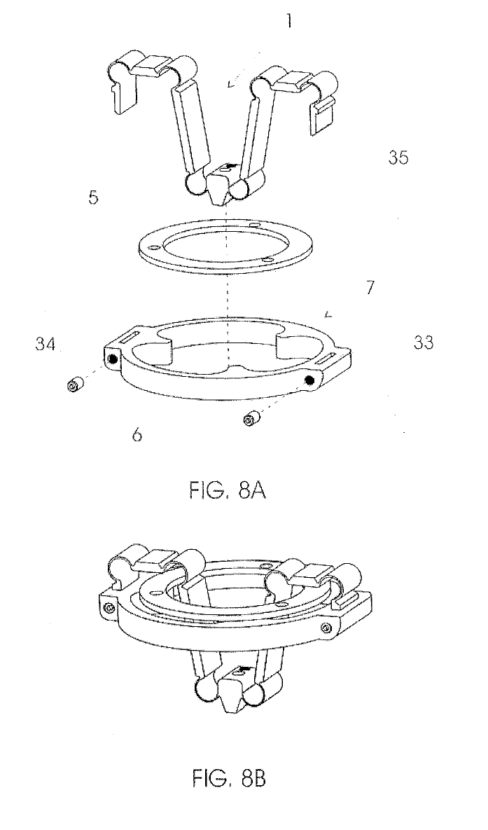

FIGS. 8A and 8B show the fastening of the compliant mechanism 1 to the holder 7. The compliant mechanism 1 has two lugs 35 which are inserted in two corresponding slots 33 formed in said holder 7, leaving the upper washer 5 between the compliant mechanism 1 and the holder 7. Said holder 7 is provided with two threaded holes 34 into which two setscrews 6, respectively, are screwed, said screws 6 firmly securing the compliant mechanism 1 to the holder 7 by pressing sideways the lugs 35. With this system for interchanging joint mechanisms 1 other kinds of said joint mechanisms 1 with different topologies and rigidities, or even other kinds of tools 200, can readily be mounted onto the micromanipulator 100.

The third component of the micromanipulator 100 is the attachment mechanism for attaching said micromanipulator to the temporal bone. It is of the uttermost importance for the proper operation of the micromanipulator 100 that it may keep unaltered its position relative to the small region where the groove 2 is to be milled.

Because of the little homogeneity of bone surface, the attachment mechanism needs to be versatile. Since it has to be used on both ears it needs also to be symmetric. And being small the region of operation, it has further to be small.

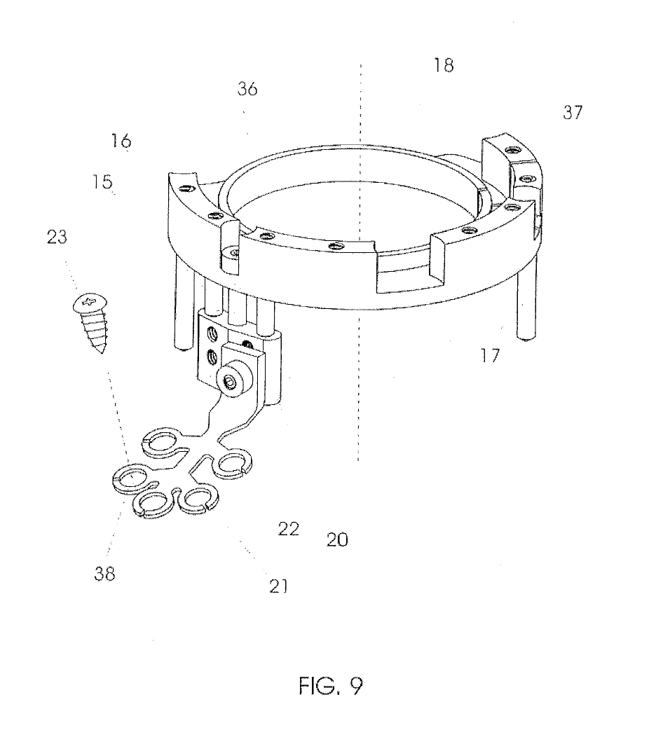

The attachment mechanism comprises (FIG. 9) a finger 21, a screw 23 for securing said finger 21 to the bone, a connector 20, a bolt 22 for securing said finger 21 to said connector 20, a bolt 16 for securing the collar 18 to the connector 20, two short setscrews 15 for setting the height of said collar 18 over said connector 20, and two long setscrews 17 for resting the collar 18 on the bone. Said collar 18 is provided with a number of threaded axial holes for housing the bolt 16 and the setscrews 15 and 17: one hole (not shown) for the bolt 16, two holes 36 for the short setscrews 15 and two he holes 37 for the long setscrews 17.

The attachment of the micromanipulator to the bone comprises the following steps:
1. After centering the micromanipulator 100, the finger 21 is fastened to the temporal bone with the screw 23. In order to be more adaptable to the uneven surface of the bone, the finger 21 has several holes 38 for inserting the screw 23.
2. With said finger 21 secured to the bone the micromanipulator has already a resting point. By loosening the bolt 22 which fastens the finger 21 to the connector 20, the micromanipulator can be rotated around the axis of said bolt 22. This movement allows the correct alignment of the micromanipulator with the ear canal.
3. By loosening the bolt 16 which fastens the connector 20 to the collar 18, screwing or unscrewing the short setscrews 15 and tightening again said bolt 16, the height of the micromanipulator can be regulated too.
4. The long setscrews 17 provide two more resting points to the micromanipulator 100. They need only be screwed at a suitable height.

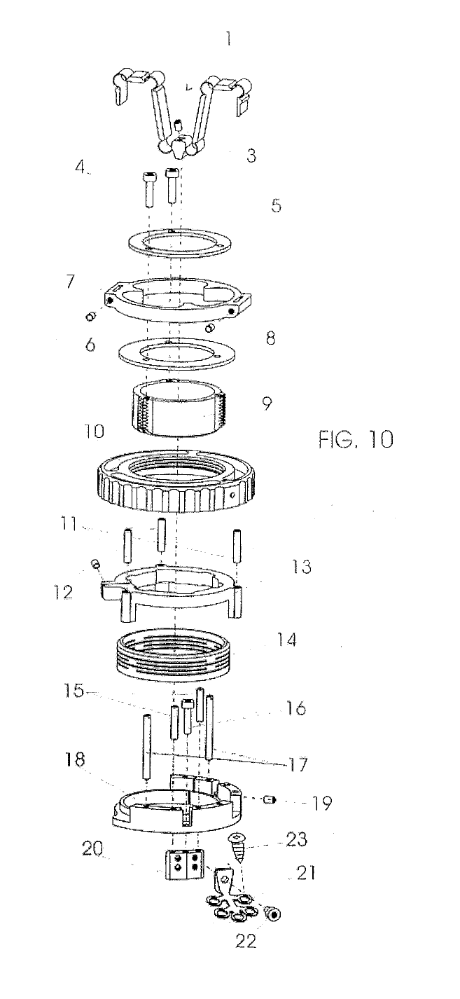

FIG. 10 shows clearly all the elements of the micromanipulator 100, exploded according to their assembled placement.

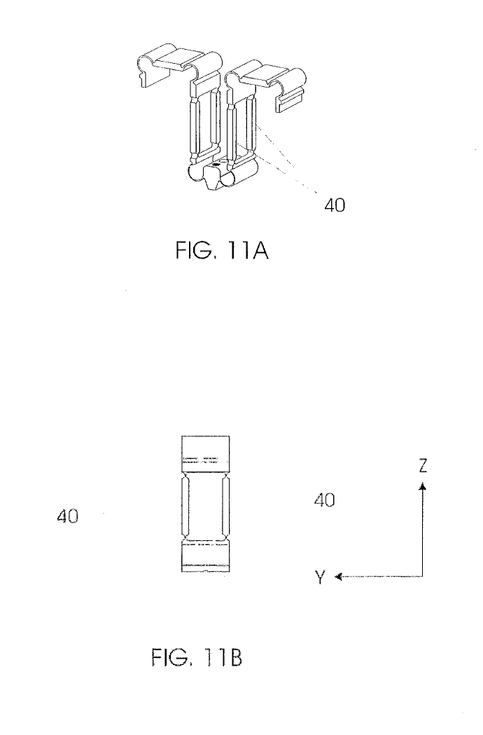

In some cases may be convenient for the compliant mechanism 1 to allow, besides the above mentioned one rotation and two translations, a further translation in the Y direction. FIGS. 11A and 11B show a compliant mechanism 1 of this type, in which each generally vertical bar is composed itself of two bars 40, this arrangement making possible a certain degree of linear movement in the Y direction.

The above description of several preferred embodiments, together with the drawings illustrating them, must not be understood as limiting the scope of the present invention, such scope being properly defined by the attached claims. Modifications and adaptations to such embodiments may be practiced without departing from said scope.

For instance, the micromanipulator 100 can be made of plastic materials and could be disposable. New topologies of compliant joints could also be designed, with other numbers of controlled degrees of freedom.

And regarding the use of the micromanipulator, it can include instruments for taking samples, local administering of drugs at selective points, electric stimulation, etc. It can particularly be used in such applications as inserting microsurgical instruments in special regions of the cochlea for taking samples for diagnosing, or as applying drugs, cells or live tissue with therapeutic purposes.

Moreover, the depth of the intervention can be increased by, for example, adding an endoscopic vision system to the micromanipulator; or a micromanipulator according to the present invention can be applied to other surgical interventions with similar requirements of accuracy.

1. A support for steadying a surgical tool handled by a surgeon comprising a limiting means for limiting the movement of said tool, characterized in that said limiting means reduces in at least two the degrees of freedom of the movement of the tool when is driven by the surgeon's hand at the moment of its use.

2. The support according to claim 1, wherein the degrees of freedom allowed by said limiting means are three translations and one rotation.

3. The support according to claim 1, wherein the degrees of freedom allowed by said limiting means are two translations and one rotation.

4. The support according to claim 1, wherein said limiting means comprises a compliant mechanism.

5. The support according to claim 4, wherein said compliant mechanism is topologically equivalent to a rigid symmetrical mechanism made of five bars and six hinged joints.

6. The support according to claim 4, wherein said compliant mechanism comprises a central segment having a hole for inserting the tool, and a blocking means for blocking said tool with respect to the compliant mechanism.

7. The support according to claim 4, wherein the compliant mechanism comprises two bars in each of the two generally vertical bars, making possible a certain degree of linear movement in the Y direction.

8. The support according to claim 4, which comprises a positioning mechanism for positioning said compliant mechanism.

9. The support according to claim 8, wherein said positioning mechanism comprises a compliant coupling formed as a tubular segment having two sets of slits positioned at 90° from each other.

10. The support according to claim 8, wherein said positioning mechanism comprises a collar which receives and secures said compliant coupling, and a clamp guide which holds said compliant coupling and is fastened to said collar.

11. The support according to claim 10, wherein said clamp guide is provided with six recesses equally spaced at 60° from each other.

12. The support according to claim 11, wherein said positioning mechanism comprises a screw-cylinder having three screwed protrusions, equally spaced at 120° from each other, and a nut-knob, so that said screw-cylinder can be inserted into said clamp guide in six different positions equally spaced at 60° from each other, and the axial position of said screw-cylinder is fine-tuned by means of said nut-knob.

13. The support according to claim 8, wherein said positioning mechanism comprises a holder for said compliant mechanism, the latter having two lugs which are inserted in two corresponding slots formed in said holder.

14. The support according to claim 8, comprising an attachment mechanism for attaching said positioning mechanism to a surface near the region to perform surgery on.

15. The support according to claim 14, wherein said attachment mechanism comprises a finger which is attached to said surface and to a connector, said collar being also attached to said connector, said attachment mechanism further comprising two short pins, which can be ascended or descended in order to regulate the height of said collar over said connector, and two long pins, which can also be ascended or descended in order to provide a good seat for said collar on said surface.

16. The support according to claim 14, wherein when performing a cochlear implant said surface is a section of the temporal bone.

17. The support according to claim 1, wherein said surgical tool is a microsurgical instrument.

18. The support according to claim 1, wherein said surgical tool is a milling cutter for milling a groove in the temporal bone.

19. The support according to claim 1, wherein said surgical tool is a micropalette or a microneedle engaged to a suction or injection system.

20. The support according to claim 1, wherein said surgical tool comprises an endoscopic vision system.
